(19) United States
(12) Patent Application Publication
(10) Pub. No.: US 2025/0111916 A1
(43) Pub. Date: Apr. 3, 2025

(54) INFORMATION PROCESSING SYSTEM, INFORMATION PROCESSING METHOD, AND NON-TRANSITORY COMPUTER-READABLE MEDIUM STORING PROGRAM

(71) Applicant: CureApp, Inc., Tokyo (JP)

(72) Inventor: Hikaru TAKEMURA, Sunnyvale, CA (US)

(73) Assignee: CureApp, Inc., Tokyo (JP)

(21) Appl. No.: 18/776,447

(22) Filed: Jul. 18, 2024

Related U.S. Application Data
(60) Provisional application No. 63/541,055, filed on Sep. 28, 2023.

(51) Int. Cl. G16H 20/00; G16H 50/30

(52) U.S. Cl. CPC G16H 20/00; G16H 50/30

(57) ABSTRACT

A technology to support improvement in effectiveness of lifestyle improvement actions is provided. An information processing system includes at least one processor, wherein the at least one processor presents at least one high-priority action category based on information about lifestyle of a target user, and presents the target user with at least one action to be practiced by the target user in relation to the presented action category.

### CROSS REFERENCE TO RELATED APPLICATIONS

This application is based on and claims priority under 35 USC § 119 to U.S. provisional patent application Ser. No. 63/541,055, filed Sep. 28, 2023, the disclosure of which is incorporated herein by reference in its entirety.

### BACKGROUND

### Technical Field

The present disclosure relates to an information processing system, an information processing method, and a non-transitory computer-readable medium storing a program.

### Related Art

Currently, companies in the United States are increasingly interested in Digital Therapeutics (DTx), which uses digital technologies to support prevention, diagnosis, and therapy of disease, and efforts are being made to reduce the prevalence of chronic diseases.

There is a growing demand for clinical evidence as companies are more interested in DTx.

Aspects of the present disclosure provide a technology to support improvement in effectiveness of lifestyle improvement actions.

### SUMMARY

An aspect of the present disclosure provides an information processing system including at least one processor, wherein the at least one processor presents at least one high-priority action category based on information about lifestyle of a target user, and presents the target user with at least one action to be practiced by the target user in relation to the presented action category.

Another aspect of the present disclosure provides an information processing method including: presenting at least one high-priority action category based on information about lifestyle of a target user; and presenting the target user with at least one action to be practiced by the target user in relation to the presented action category.

Still another aspect of the present disclosure provides a non-transitory computer readable medium storing a program that causes a computer to execute an information processing method including: presenting at least one high-priority action category based on information on lifestyle of a target user; and presenting the target user with at least one action to be practiced by the target user in relation to the presented action category.

According to the aspects of the present disclosure, it is possible to support improvement in effectiveness of lifestyle improvement actions.

### BRIEF DESCRIPTION OF DRAWINGS

Exemplary embodiments of the present invention will be described in detail based on the following figures, wherein:

FIG. 1 illustrates a configuration example of an information processing system assumed in Exemplary embodiment 1;

FIG. 2 illustrates a hardware configuration of a support system;

FIG. 3 is a sequence diagram illustrating an overview of support services assumed in Exemplary embodiment 1;

FIG. 4 illustrates a display example of questions related to a lifestyle;

FIG. 5 shows an example of a screen showing results of analyzing a lifestyle of a target user by each action category;

FIG. 6 shows an example of a table used for rank estimation of scores calculated by each action category;

FIG. 7 shows an example of a table used to select a featured category;

FIG. 8 shows an example of a selection screen for effective action candidates for lifestyle improvement in the featured category; and

FIG. 9 is a sequence diagram illustrating an overview of support services assumed in Exemplary embodiment 2.

### DETAILED DESCRIPTION

Hereinafter, exemplary embodiments of the present disclosure will be described with reference to the drawings.

### Exemplary Embodiment 1

### <System Configuration>

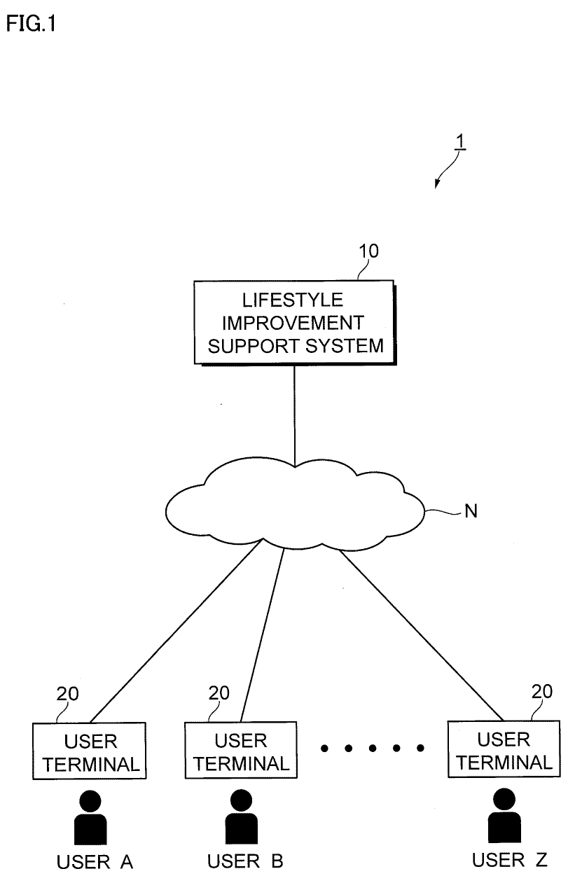

FIG. 1 illustrates a configuration example of an information processing system 1 assumed in Exemplary embodiment 1.

The information processing system 1 shown in FIG. 1 includes a lifestyle improvement support system (hereinafter referred to as “support system”) 10 that provides lifestyle improvement support service (hereinafter referred to as “support service”), user terminals 20 each operated by a user of the support services, and a network N that connects these terminals.

Note that the support system 10 is an example of an information processing system in a narrow sense.

The support system 10 in the exemplary embodiment is a system that distributes applications that support lifestyle improvement (hereinafter referred to as “support app”), stores user data entered through the support app, and provides information to support lifestyle improvement.

The support system 10 in the exemplary embodiment is operated by a company or organization where a user works. However, an enterprise or the like entrusted by the company or organization where the user works may operate the support system 10.

The support app in the exemplary embodiment is installed on the user terminal 20 and is used to record the daily life of the user. A record of daily life includes, for example, blood pressure values, weight, pulse, history of activity, and a record of feelings and moods.

The user terminal 20 is a computer terminal on which the support app is installed. The user terminal 20 in the exemplary embodiment is, for example, a smart phone, a tablet-type computer, a laptop-type computer, and a desktop-type computer.

The user terminal 20 can communicate with the support system 10 through the network N. The user terminal 20 uploads the user data entered through the support app to the support system 10, and the support services are provided by the support system 10.

The network N is, for example, a local area network (LAN), the Internet, or a mobile communication system (4G, 5G).

### <Hardware Configuration>

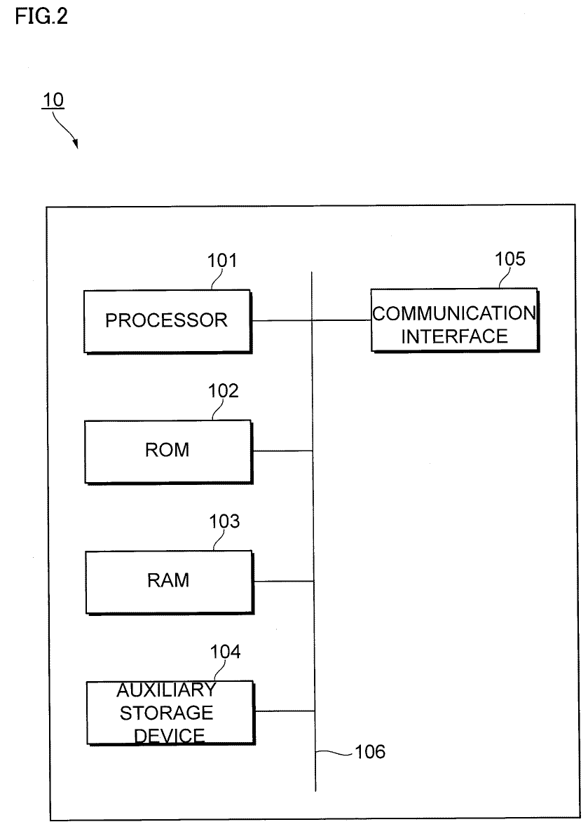

FIG. 2 illustrates a hardware configuration of the support system 10. The support system 10 is configured with, for example, one or more servers.

The support system 10 includes a processor 101, a Read Only Memory (ROM) 102 storing a Basic Input Output System (BIOS), etc., a Random Access Memory (RAM) 103 used as a work area of the processor 101, an auxiliary storage device 104, and a communication interface 105. Each device is connected via a bus and other signal wires 106.

The processor 101 is a device that implements various kinds of functions through the execution of programs. In the exemplary embodiment, the support app and Operating System (OS) are collectively called programs.

The processor 101, ROM 102, and RAM 103 function as a computer.

The auxiliary storage device 104 is configured with, for example, a hard disk device or a semiconductor storage.

The communication interface 105 is an interface for communicating with other servers or user terminals 20 (refer to FIG. 1) via the network N. The communication interface 105 (registered trademark), Wi-Fi (registered trademark), mobile complies with Ethernet communication systems and other communication standards.

### <Data Stored in Auxiliary Storage Device>

The auxiliary storage device 104 stores user data collected through the support app in addition to the support app.

By the way, the support app collects user data through operation and entry by the user and communication with a wearable device that the user wears. For example, blood pressure value, heart rate, exercise, sleep time, etc. are sequentially uploaded from the connected wearable device to the support app. In addition, the dietary content and alcohol intake may be collected through image analysis.

The user data includes, for example, user attributes (gender, date of birth, age, height, weight), blood pressure values, activity records, mood records, physical condition records, medical examination history, operation history of the support app, biological characteristics, psychological characteristics, social characteristics, habits, goal achievement status, featured categories presented by the support services, recommended actions for featured categories, contents that encourage the practice of the actions (including information that identifies the contents), information that motivates the continuous practice of the actions, records of proficiency with the contents, etc. However, the user data does not need to be all of these information, but may be part thereof.

The blood pressure values are, for example, systolic blood pressure (i.e., the highest blood pressure value) and diastolic blood pressure (i.e., the lowest blood pressure value).

The activity records include, for example, usage history of the support app, medications records, dietary records, smoking records, drinking records, and exercise records. The activity records are an example of information about activities.

The medications records include, for example, the content and number of medications taken, and the time at which the medications were taken. The medications records are an example of information about medications.

The dietary records include, for example, the content of the meals consumed, and the amount of salt taken. The dietary records are an example of information about diet.

The smoking records include, for example, the presence or absence of smoking, the date and time of smoking, and the number of cigarettes smoked. The smoking records are an example of information about smoking.

The drinking records include, for example, the date and time of drinking, the amount of alcohol intake, and the type of alcohol. The drinking records are an example of information about drinking.

The exercise records include, for example, records related to sports as well as records of activities in daily life such as walking, shopping, and cleaning. The exercise records are an example of information about exercise.

The mood records include, for example, a perceived mood. The mood records are an example of information about mood.

The physical condition records include, for example, a perceived physical condition or symptoms. The physical condition records are an example of information about physical condition.

The medical examination history includes, for example, the start date of the therapy, the date of the consultation, the details of the therapy, and advice from the doctor or other healthcare professionals. The medical examination history is an example of information about medical examination.

The operation history of the support app includes, for example, the operation history of the activation operation, the entry operation of blood pressure values and reflection, the operation to answer the questions presented by the support service, and the operation related to the selection of the actions to be practiced.

The biological characteristics include, for example, the presence or absence of other diseases, the presence or absence of injuries during therapy, the presence or absence of knee or foot pain, the presence or absence of experience in hypertensive therapy, the number of years since the high blood pressure was pointed out, the intensity of seasoning at home, and the amount of intake of food.

The psychological characteristics include, for example, expectations for the support app, willingness to acquire knowledge and information that can contribute to improve lifestyle, feeling that salt reduction is challenging, feeling inability to alter the sense of taste, and a psychological resistance to leaving a meal.

The social characteristics include, for example, wake time, bedtime, work pattern (such as shifts, day duty, and night duty), days of the week to work, the start time of the work, the time to return home, regular days off, and the presence or absence of a heater in a locker room.

The habits include, for example, an exercise habit, a weight measurement habit, a habit of calorie checking on food labels, a habit of low-fat food choices, a habit of not taking caffeine after 4 p.m., a habit of eating and drinking after 10 p.m., a habit of skipping breakfast, a habit of eating between meals, a habit of bathing one hour before bedtime, a habit of stretching and massaging before bedtime, and a habit of sleeping for more than six hours.

The goal achievement status includes, for example, the relationship between the user's status and the goal set by the doctor, etc. for each user.

Note that the above-described information can be classified into subjective information and objective information.

The featured categories presented by the support service are, for example, action categories from among diet, exercise, weight loss, sleep, stress, and alcohol that have a higher possibility of contributing to the improvement of the target user's lifestyle than others. In other words, the featured category is an example of a high-priority action category.

Note that the featured categories are determined based on, for example, answers to questions to the target user regarding his/her lifestyle habits and preferences, and medical evaluation criteria, and notified to the target user by the support service. Review and update of the featured categories are carried out periodically (for example, every month) in response to the status of lifestyle improvements.

The recommended actions for featured categories (hereinafter also referred to as “recommended actions”) are actions selected from a group of candidates for actions related to a featured category. For example, in the case where the featured category is “exercise,” the candidates for the actions include walking (total distance, total length of time, etc.), jogging (distance, total length of time, etc.), housework (type, length of time by type, total length of time, etc.), cycling (total distance, total length of time, etc.), swimming (total distance, total length of time, etc.), ball games (type, total length of time, etc.), and dance (total length of time).

Candidates for the recommended actions are determined based on, for example, a history of practice by the target user and a record of improvement effects after practice. In the exemplary embodiment, the target user selects the recommended action. The review and update of the recommended actions to be practiced are carried out in a shorter cycle (for example, every week) than the review and update of the featured categories.

The contents that encourage the practice of the actions (including information that identifies the contents) refer to, for example, learning contents that explain how to practice recommended actions or expected effects of practicing recommended actions.

The information that motivates the continuous practice of the actions is, for example, points or other rewards provided for the continued practice of the target user. This type of information includes, for example, accumulated point increases, object advancement, growth, and evolution.

The proficiency with the content is the target user's level of understanding as evaluated through the target user's responses and actual performance. The proficiency is evaluated in the lifestyle improvement support service and notified to the target user.

In addition, the user data includes user data collected from the target user in relation to the support service currently in operation, as well as user data collected from the target user in relation to other support services.

### <Processing Sequence Executed by Information Processing System>

### <Overview of Support Service>

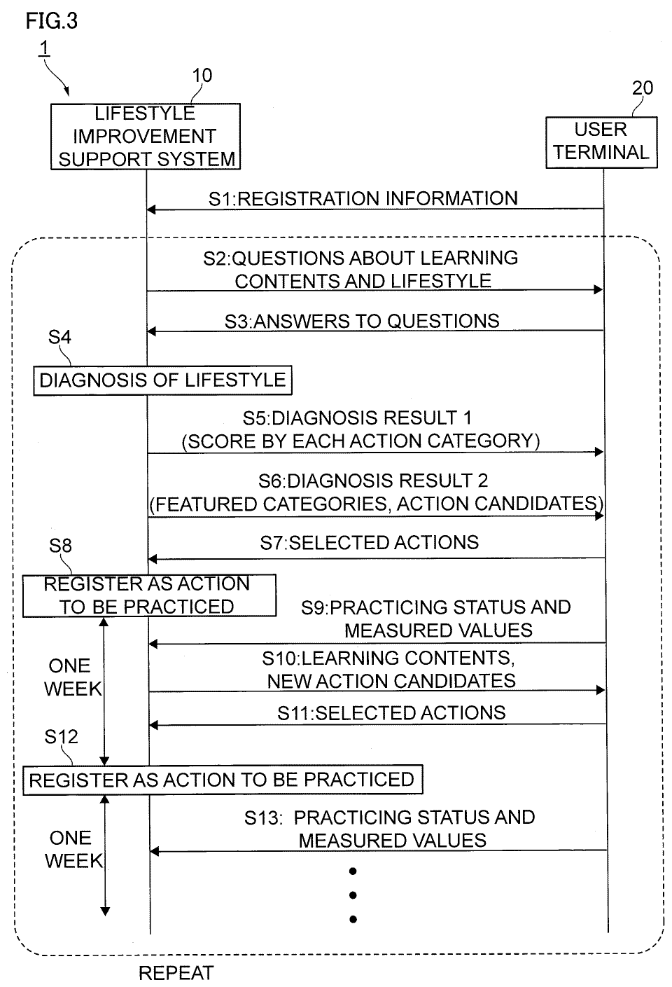

FIG. 3 is a sequence diagram illustrating an overview of a support service assumed in Exemplary embodiment 1.

The processing sequence shown in FIG. 3 is implemented through the execution of programs by the processor 101, which constitutes the support system 10 (see FIG. 2), and a not-shown processor, which constitutes the user terminal 20. The symbol S shown in the figure represents a step.

### [Step 1]

In the case of FIG. 3, the user registration information is transmitted from the user terminal 20 to the support system 10. For example, the measurement values of blood pressure and pulse, and information about medications taken are transmitted as the registration information. In the case of the exemplary embodiment, the entry and transmission of the registration information is conducted through the support app.

### [Step 2]

The support system 10 transmits questions about learning contents and lifestyle to the user terminal 20.

The learning contents include knowledge and information that contribute to the improvement of lifestyle. The learning contents are prepared for each disease, for example. In the case of the exemplary embodiment, high blood pressure is assumed as a disease. Note that the learning contents may be text-based, static-image-based, moving-image-based, or speech-based.

The questions related to the lifestyle are questions about the habits and preferences in daily life of each user. The questions in the exemplary embodiment are prepared to include, for example, six action categories of diet, exercise, weight loss, sleep, stress, and alcohol.

The questions related to the lifestyle are composed of questions by each action category. In the case of the exemplary embodiment, the total number of questions is, for example, 50.

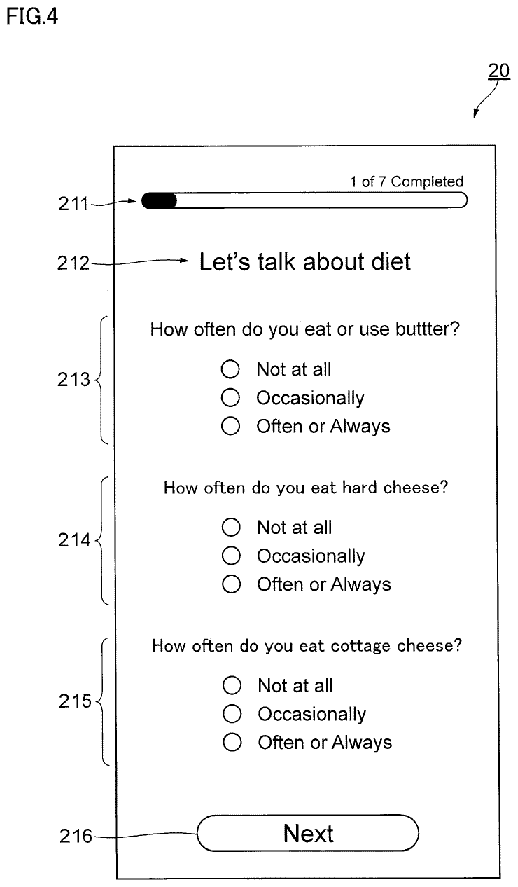

FIG. 4 illustrates a display example of questions related to a lifestyle.

The questions in FIG. 4 are shown on the display of the user terminal 20. As described above, the total number of questions in the exemplary embodiment is 50. For this reason, only part of the questions is shown in FIG. 4.

In the question screen shown in FIG. 4, a progress display section 211, a title section 212, question sections 213, 214, and 215, and a “Next” button 216 are placed. Needless to say, the placement shown in FIG. 4 is an example, and the number of questions to be displayed varies depending on the screen size, font size, etc. of the display.

The progress display section 211 is used to display the current progress against the total number of questions. In FIG. 4, the progress is represented by the length of the bar chart and the text (for example, “1 of 7 completed”). The length of the colored part of the bar chart indicates the progress. In the case of FIG. 4, the length of the colored part of the bar chart is roughly 1/7 of the total length.

The title section 212 is used to display the name of the action category to which the question relates. The action category assumed in FIG. 4 is “Diet.” Therefore, the title section 212 displays “Let's talk about diet.”

The question section 213 displays a question about the frequency of butter intake, and there are three options for responding to the question. The same is true for other questions.

Note that the three options are in the form of radio buttons. Therefore, only one of the three options can be selected. The same is true for other questions to be described later.

The question section 214 displays a question about the frequency of hard cheese intake, and there are three options for responding to the question.

The question section 215 displays a question about the frequency of cottage cheese intake, and there are three options for responding to the question.

In the case of FIG. 4, when the “Next” button 216 is operated, the screen changes to the next page.

### [Step 3]

Return to the description of FIG. 3.

The user terminal 20 transmits the user's answer to the question to the support system 10. In the case of the exemplary embodiment, the answer is entered through the selection of options by the user.

### [Step 4]

The support system 10 diagnoses the lifestyle of the target user based on the answers received. In the case of the exemplary embodiment, for example, scores are calculated for each of the six action categories described above.

In the case of the exemplary embodiment, the score is calculated as the sum of the points assigned to the user's answer (selection of options) to each question. For example, the best lifestyle option of the three is assigned with 1 point, the second best lifestyle option of the three is assigned with 2 points, and the worst lifestyle option of the three is assigned with 3 points.

The support system 10 in the exemplary embodiment calculates a score for each action category.

### [Step 5]

The support system 10 presents scores for each action category to the user terminal 20 as a result of analyzing the lifestyle of the target user.

In the case of the exemplary embodiment, as a result of analyzing the lifestyle, the score Sn (n=1, 2, 3, 4, 5, 6) calculated for each of the six action categories is presented in the form of a bar chart.

The score S1 corresponds to “Diet,” the score S2 corresponds to “Exercise,” the score S3 corresponds to “Weight,” the score S4 corresponds to “Sleep,” the score S5 corresponds to “Stress,” and the score S6 corresponds to “Alcohol.”

In this case, the length of the bar chart Ln corresponding to the score Sn (where n=1, 2, 3, 4, 5, 6) is calculated based on the following equation.

$$Ln [ \% ] = 150 - \{ ( 50 / ( number of questions ) ) * Sn \}$$

For example, if it is assumed that the weight loss score S3 is “15” and the number of questions related to weight loss is “10,” the length of the bar chart L3, which represents the result of analyzing the lifestyle related to weight loss, is calculated to be 75% (=150−{(50/10)*15}).

In this connection, if the weight loss score S3 is “10” (when each answer in all 10 questions is assigned with 1 point), the length of the bar chart L3 is calculated to be 100% (=150−{(50/10)*10}), if the weight loss score S3 is “20,” the length of the bar chart L3 is calculated to be 50% (=150−{(50/10)*20}), and if the weight loss score S3 is “30” (when each answer in all 10 questions is assigned with 3 point), the length of the bar chart L3 is calculated to be 0% (=150−{(50/10)*30}).

In the case of the exemplary embodiment, the lower the score Sn, the better the analysis results of the lifestyle, and the higher the score Sn, the worse the analysis results of lifestyle.

Therefore, an action category with a bar chart length of 100% means that the lifestyle is good, and an action category with 0% means that the lifestyle is bad.

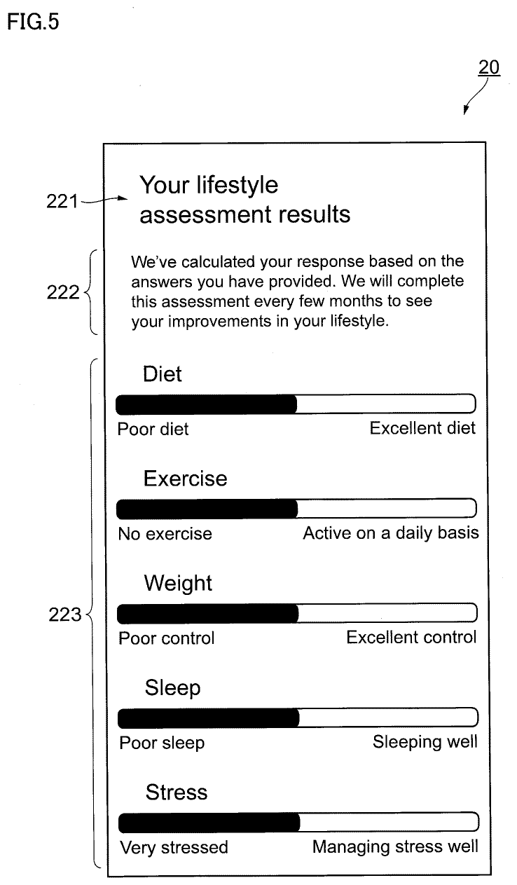

FIG. 5 shows an example of a screen showing results of analyzing a lifestyle of a target user by each action category.

The screen in FIG. 5 is shown on the display of the user terminal 20.

The screen shown in FIG. 5 includes a title 221, a description section 222 for the analysis results, and an analysis result display section 223. Needless to say, the arrangement of sections shown in FIG. 5 is merely an example.

In the case of FIG. 5, the title 221 states “Your lifestyle assessment results.”

In the case of FIG. 5, the description section 222 states that the analysis results were calculated based on the user's answers, and that lifestyle is analyzed every few months to see the improvement of lifestyle. Of course, the text is merely an example. In the exemplary embodiment, the lifestyle of the target user is re-assessed every month.

In the case of FIG. 5, in the analysis result display section 223, the analysis results for five of the six action categories are represented by the bar chart lengths Ln. Note that the analysis result for one remaining action category (that is, alcohol) can be seen by scrolling the screen.

In the case of FIG. 5, the bar chart is represented as the length in the horizontal direction. As described above, the shorter the length of the bar chart, the worse the analysis results, and the longer the length of the bar chart, the better the analysis results.

For this reason, the far right of the bar chart has a description indicating the good state, and the far left of the bar chart has a description indicating the bad state. For example, for “Diet,” there is a description “Excellent diet” on the far right of the bar chart, and “Poor diet” on the far left of the bar chart.

By observing the length of the bar chart corresponding to each action category, the target user can confirm the status of his/her lifestyle for each action category.

In other words, the target user can confirm at a glance if his/her lifestyle is good or bad in which action category.

To put it another way, it is possible to grasp the analysis results of lifestyle related to the six action categories from a higher perspective.

As a result, the target user can notice the action categories that should be focused on to improve his/her lifestyle.

### [Step 6]

Return to the description of FIG. 3.

As a result of analysis of the lifestyle of the target user, the support system 10 presents the featured categories and the candidate actions to be practiced on the user terminal 20.

The support system 10 determines the featured categories based on the evaluation results of the score Sn calculated in step 5 and the medical evaluation criteria.

First, based on the evaluation criteria prepared for each action category, the support system 10 classifies the calculated score Sn as any of “Rank A (Good)”, “Rank B (Intermediate)”, and “Rank C (Bad)”.

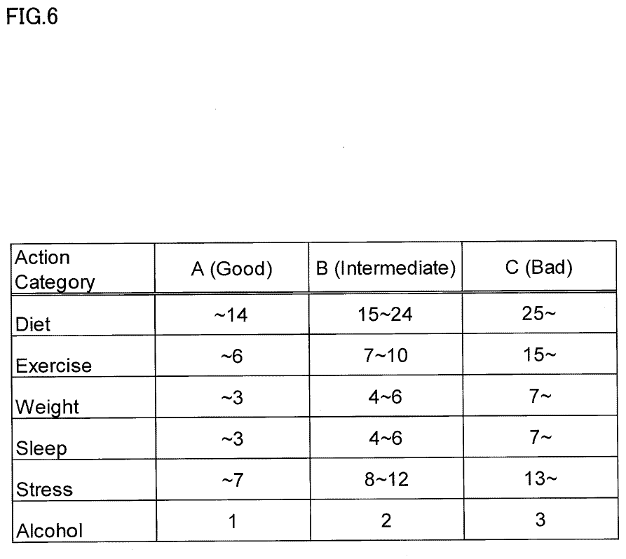

FIG. 6 shows an example of a table used for rank estimation of scores calculated by each action category.

In the case of FIG. 6, six action categories are assigned to the leftmost column, a range of scores classified as the rank A is assigned to the first column from the left to the right, a range of scores classified as the rank B is assigned to the second column from the left to the right, and a range of scores classified as the rank C is assigned to the rightmost column.

As shown in FIG. 6, the range of scores assigned to the rank A, B, or C differs depending on the action category.

For example, in the case of “Diet,” the score of S1 of 14 or less is classified as the rank A, the score of S1 of 15 or more and 24 or less is classified as the rank B, and the score of S1 of 25 or more is classified as the rank C.

In the case of “Exercise,” the score of S2 of 6 or less is classified as the rank A, the score of S2 of 7 or more and 10 or less is classified as the rank B, and the score of S2 of 15 or more is classified as the rank C.

As shown in FIG. 6, the range of scores assigned to each rank varies in response to the action category. One of the reasons is the difference in the number of questions about each action category.

For example, there is only one question about “Alcohol.” Therefore, the rank A, B, or C is determined by which one of the three options has been selected.

Once the rank is determined by each action category, the support system 10 selects the featured categories from among the six action categories. In the case of the exemplary embodiment, the number of featured categories is three.

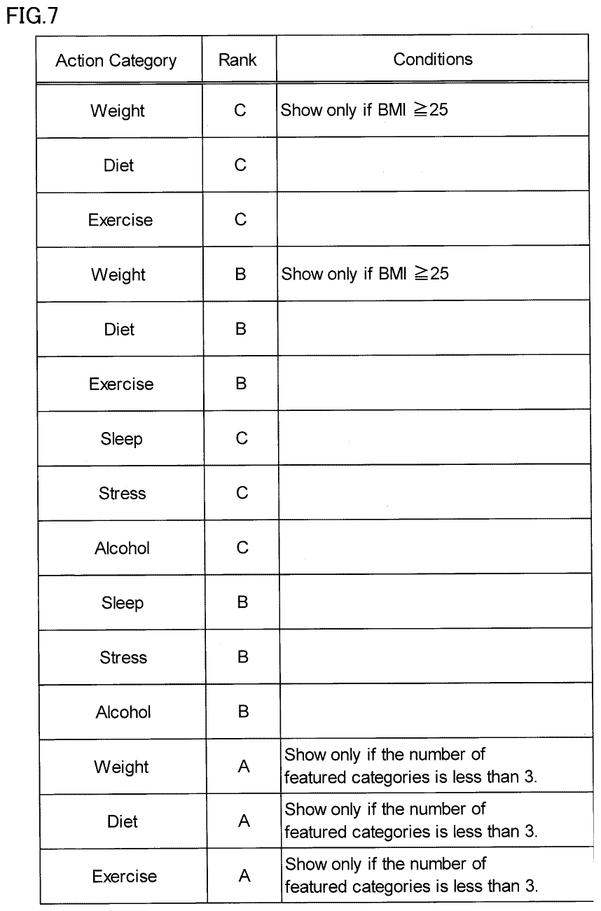

FIG. 7 shows an example of a table used to select the featured categories. The table example shown in FIG. 7 gives priority relationship to the combination of action category and rank based on medical evaluation criteria. In this regard, the table example shown in FIG. 7 is also an example of actual performance data of users other than the target user.

In the case of FIG. 7, the action category is placed in the leftmost column, the rank information is placed in the middle column, and the conditions required for the case selecting the featured category are placed in the rightmost column.

In the table shown in FIG. 7, the order to be selected as a featured category is determined by the combination of the action category and rank. The table represents that the higher the position, the higher the priority.

First, the support system 10 confirms whether the score S3 calculated for the “Weight” of the target user is the rank C based on the table shown in FIG. 7.

If the score S3 corresponding to the “Weight” of the target user is C, the support system 10 further determines whether a Body Mass Index (BMI) of the target user is 25 or more. If the BMI is 25 or more, the support system 10 selects the “Weight” in the action category as the first featured category. Note that the BMI can be calculated based on the weight and height of the user data.

However, if the score S3 corresponding to the “Weight” of the target user is the rank C, but the BMI is less than 25, or if the score S3 corresponding to the “Weight” of the target user is the rank A or B, the support system 10 confirms whether the score S1 calculated for the “Diet” of the target user is the rank C.

Similarly, the rank of the action category of the target user is checked against the table shown in FIG. 7 until three featured categories are selected.

Note that, if the total number of featured categories is less than three, “Weight,” “Diet,” or “Exercise,” may possibly be selected as a featured category even though the corresponding score is the rank A.

Once the three featured categories are determined, the support system 10 presents the three featured categories to the user terminal 20. The three featured categories may be presented on the user terminal 20 in a view format, or may be presented in association with the selection screen for actions to be practiced.

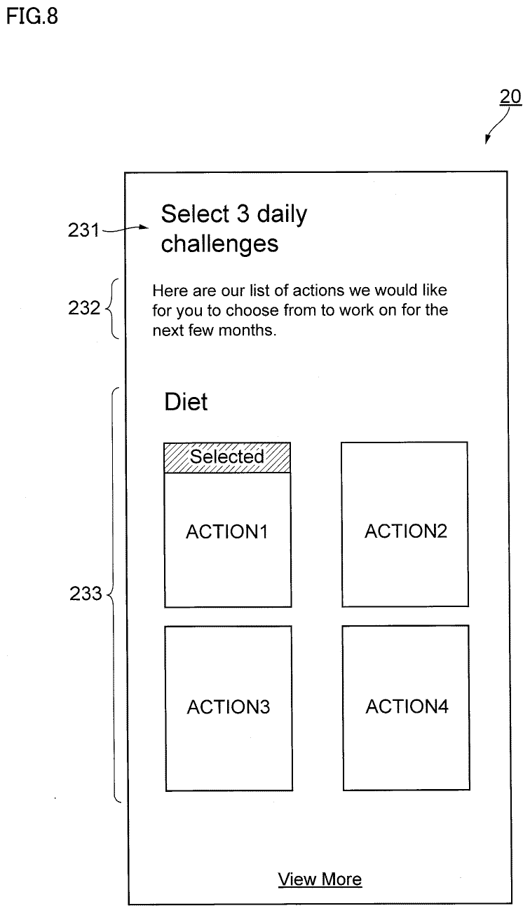

FIG. 8 shows an example of a selection screen for effective action candidates for lifestyle improvement in the featured category.

The screen in FIG. 8 is shown on the display of the user terminal 20.

The screen shown in FIG. 8 includes a title 231, a description section 232, and an action candidate section 233 for each featured category. Needless to say, the arrangement of sections shown in FIG. 8 is merely an example.

In the case of FIG. 8, the title 231 shows “Select 3 daily challenges.” Note that the title 231 also represents the operation required of the target user.

In the case of FIG. 8, the description section 232 contains a description of the presented action candidates.

The action candidate section 233 for each featured category presents the action candidates to be practiced for each featured category in a selectable manner.

Note that the action candidates to be practiced are presented in order based on the priorities among the three featured categories. The priority is determined based on the table shown in FIG. 7. The priority is determined by the relative order in the table.

In the case of FIG. 8, the “Diet,” which has the highest priority of the three featured categories, is presented as the first featured category.

FIG. 8 presents four action candidates to be practiced, from the “Action 1” to “Action 4.” If any of the four action candidates is tapped, the corresponding candidate is brought into a selected condition or unselected condition.

Note that, depending on the featured category, the number of action candidates to be presented may be two, three, or five or more.

In addition, the action candidate previously selected by the target user may be excluded from the display subject for a certain period of time. This function can provide an environment in which actions to be selected are difficult to be fixable.

As a result, the target user selects and practices various actions, which makes it easier to continuously work toward the improvement of his/her lifestyle compared to practicing only the same actions.

In addition, even in the case where the same action category is repeatedly presented as a featured category, it is also possible to give the target user the pleasure of selecting the candidate by making it difficult for the action candidates to be practiced being fixable.

In the case of the exemplary embodiment, the action candidates are selected in a checkbox format. Therefore, it is possible to select two candidates for one featured category.

FIG. 8 represents a condition in which the “Action 1” is selected by the target user. Note that, if the “Action 1” in the selected condition is tapped again, the selected condition is canceled. The action candidates include, for example, “Refraining from drinking beverages containing carbonate or sugar.”

Note that, when “View more” at the bottom of the screen is tapped, the action candidates to be practiced for the remaining two featured categories are presented in the selectable manner.

For example, multiple action candidates to be practiced are presented for “Exercise” or “Sleep.”

For example, in the case where an action candidate is selected for each featured category, one action is selected for “Diet,” one action is selected for “Exercise,” and one action is selected for “Sleep.”

Note that, in the case where multiple action candidates are selected for one featured category, for example, two actions are selected for “Diet” and one action is selected for “Exercise.”

When the third action is selected, the user terminal 20 presents a screen for confirming the three selected actions to the user. If the user agrees to the three actions presented, the user's selection of actions is completed.

### [Step 7]

Return to the description of FIG. 3.

Once the user's selection of three actions is finalized, the user terminal 20 transmits the actions selected by the user to the support system 10.

### [Step 8]

The support system 10 that has received the three actions registers the received three actions as the actions to be practiced.

### [Step 9]

The user terminal 20 transmits the practicing status of actions and measured values collected through the support app to the support system 10. Note that the user terminal 20 may transmit the collected user data to the support system 10 as it is.

The transmission of the practicing status etc. here may be executed at a pre-scheduled time (for example, every noon, every hour), or when the user accesses the support system 10 by operating the user terminal 20.

### [Step 10]

The support system 10 notifies the user terminal 20 of learning contents and new action candidates related to the featured category.

The learning contents include knowledge and information that contribute to the improvement of lifestyle. By the presentation of learning contents, the practice of actions by the user is promoted and improvement of lifestyle can be expected compared to the case where only the practice of actions is required. The learning contents here are examples of contents that encourage users to practice actions.

The new action candidates are presented from among the action candidates related to the featured categories. In the case of the exemplary embodiment, the action candidates selected by the user within a certain period of time are excluded. This presents new actions to the user as selection candidates.

Note that the presentation of the learning contents and the presentation of the new action candidates may be carried out separately.

### [Step 11]

Similar to Step 7, once the user's selection of three actions is finalized, the user terminal 20 transmits the actions selected by the user to the support system 10.

### [Step 12]

Similar to Step 8, the support system 10 that has received the three actions registers the received three actions as the actions to be practiced.

In the case of the exemplary embodiment, the period from Step 8 to Step 12 is one week.

### [Step 13]

Similar to Step 9, the user terminal 20 transmits the practicing status of actions and measured values collected through the support app to the support system 10.

Hereinafter, the processing operations corresponding to Steps 6 to 9 and Steps 10 to 13 are executed every week.

Note that, in the case of the exemplary embodiment, the processes from Step 2 are carried out repeatedly every month. In other words, the featured categories are reviewed and updated on a monthly basis, and the action candidates to improve the updated featured categories are presented and put into practice repeatedly.

### Brief Conclusion

According to the information processing system 1 (refer to FIG. 1) by the exemplary embodiment, it is possible to support the practice of actions related to the featured categories specified through the analysis of the lifestyle of the target user. As a result, the effectiveness of actions that improve lifestyle is increased.

In addition, by leaving the actions to be practiced to the user's own selection, it is possible to increase the likelihood that actions improving lifestyle are practiced.

Moreover, by reviewing and updating the featured categories every month and reviewing and updating the actions related to the featured categories every week, it is easier to practice the actions required of the target user. As a result, the improvement effect of the featured categories can be enhanced.

### Exemplary Embodiment 2

In this exemplary embodiment, a description will be given of a case where the support system 10 (refer to FIG. 1) determines the actions to be practiced for the featured categories and presents the determined actions to the target user.

Note that the system configuration and the like in the exemplary embodiment are the same as those in Exemplary embodiment 1.

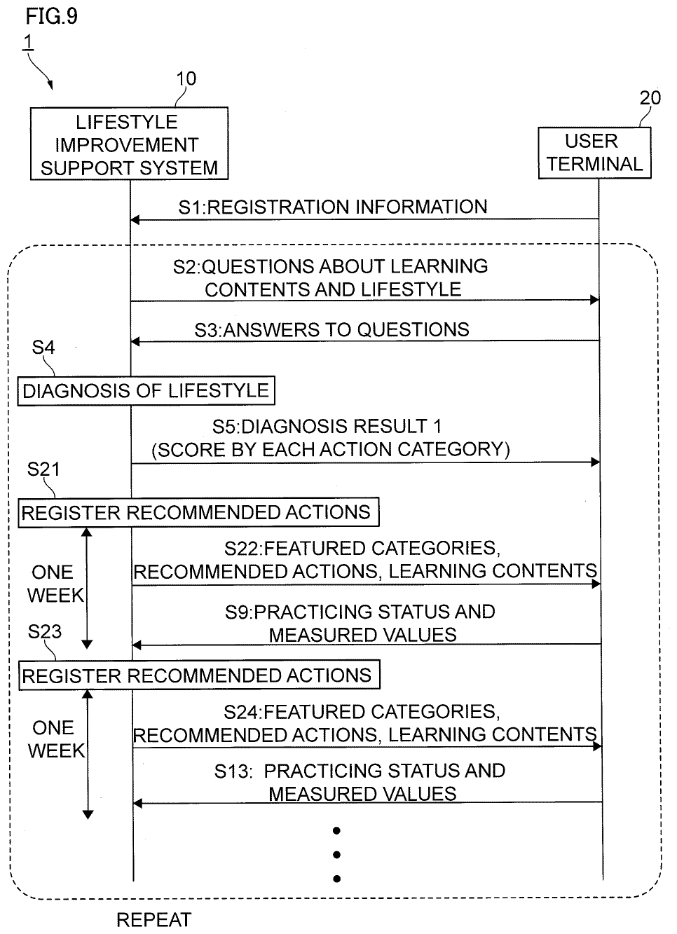

FIG. 9 is a sequence diagram illustrating an overview of a support service assumed in Exemplary embodiment 2. In FIG. 9, components corresponding to those of FIG. 3 are provided with same reference signs.

As shown in FIG. 9, the processing operations in Step 1 to Step 5 are the same as those in Exemplary embodiment 1.

### [Step 21]

The support system 10 registers actions that should be practiced by the target user (recommended actions) among the action candidates related to the featured category.

In the case of the exemplary embodiment, the number of featured categories is also three.

In addition, the action candidates are prepared for each action category in advance, similar to Exemplary embodiment 1.

The support system 10 in the exemplary embodiment registers one recommended action for one featured category, for example, according to the priority predetermined for the actions related to the action category. In other words, the support system 10 determines the action that should be practiced in the next week without presenting selection candidates to the user.

The priority is determined based on, for example, the preference of actions or actual performance of practice in a user group with a high attribute similarity with the target user.

### [Step 22]

The support system 10 notifies the user terminal 20 of the featured categories, recommended actions, and the learning contents.

### [Step 9]

The user terminal 20 transmits the practicing status of actions and measured values collected through the support app to the support system 10.

### [Step 23]

When one week has passed since the recommended actions were registered in Step 21, or the next one week approaches, the support system 10 registers new recommended actions.

### [Step 24]

The support system 10 notifies the user terminal 20 of the featured categories, recommended actions, and the learning contents.

### [Step 13]

The user terminal 20 transmits the practicing status of actions and measured values collected through the support app to the support system 10.

Hereinafter, the processing operations corresponding to Steps 21, 22, and 9 and Steps 23, 24, and 13 are executed every week.

In the case of the exemplary embodiment, the processes from Step 2 are also carried out repeatedly every month. In other words, the featured categories are reviewed and updated on a monthly basis, and the recommended actions to improve the updated featured categories are presented and put into practice repeatedly.

### Brief Conclusion

In the case of the information processing system 1 (refer to FIG. 1) by the exemplary embodiment, since the support system 10 determines the recommended actions and presents thereof to the target user, the burden on users, who find the selection of actions every week troublesome, can be reduced.

In addition, the recommended actions are determined based on the preference of actions or actual performance in practice in a user group with a high attribute similarity with the target user; thereby effectiveness of lifestyle improvement can be enhanced.

### Other Exemplary Embodiments

(1) So far, the exemplary embodiments of the present disclosure have been described, but the technical scope of the present disclosure is not limited to the scope of the above-described exemplary embodiments. Various modifications or improvements added to the above-described exemplary embodiments may be apparently included in the technical scope of the present disclosure from the description of the claims.

(2) The processor in the above-described exemplary embodiments refers to a processor in a broad sense including, in addition to a general-purpose processor (for example, a Central Processing Unit (CPU)), a dedicated processor (for example, a Graphical Processing Unit (GPU), an Application specific Integrated Circuit (ASIC), a Field Programmable Gate Array (FPGA), a program logic device, etc.).

Moreover, the operation of the processor in each of the above-described exemplary embodiments is not limited to a single processor, but may be performed in cooperation by multiple processors. In addition, the order of execution of each operation in the processor is not limited to the order described in each of the above-described exemplary embodiments, but may be changed individually.

(3) In the above-described Exemplary embodiment 1, the number of answers (options) to each question is standardized to three, but the number of answers (options) to each question may all be two, four, or more.

(4) In the above-described Exemplary embodiment 1, the number of answers (options) to each question is standardized, but the number of answers (options) may vary by each question.

For example, a question with two answers (options) or a question with four answers (options) may be included.

(5) In the above-described exemplary embodiments, three featured categories are selected from among the six action categories, but the number of action categories is not limited to six, and the number of featured categories is not limited to three.

For example, the number of action categories may be five or less or seven or more. In addition, the number of featured categories may be one, two, four, or more.

(6) In the above-described exemplary embodiments, the rank of each action category is determined using the table shown in FIG. 6, and the featured category is determined by comparing the rank of the determined action category with the table shown in FIG. 7, but the featured category may be determined based on the relative rank relationships among the six action categories.

For example, the featured category may be determined by prioritizing the rank B over the rank A, and prioritizing the rank C over the rank B. In addition, if there are more than four or more C-ranked action categories, for example, three featured categories may be determined by random drawing.

(7) In the above-described Exemplary embodiment 1, selection of three actions from among the action candidates related to the featured category is required, but it may be possible to finish the selection operation by, for example, selecting two actions.

For example, if the user is not in a good physical or psychological condition, even if the user selects the three actions forcibly, it is unlikely that the user will put the actions into practice. Rather, the selection of only the number of actions that the target user considers practicable leads to expectation for continuous practice.

In addition, in daily life, there are instances to get sick, injured, or suddenly get sick. In such cases, it may be possible to change the actions or suspend the practice within the practice period (for example, a week).

For example, in the case of a leg injury, the action to be practiced may be changed from walking to upper body exercise or stretching.

(8) In the above-described exemplary embodiments, the actions to be practiced are reviewed and updated on a weekly basis and the featured categories are reviewed and updated on a monthly basis, but the cycle of the review and update is not limited thereto. For example, the actions to be practiced may be reviewed and updated every two weeks, or may be carried out when the featured categories are reviewed and updated. Similarly, the review and update cycle for the featured categories may not be limited to one month, but may be, for example, two months.

(9) In the above-described exemplary embodiments, it is assumed that the operator of the support system 10 is a corporation, an organization, etc., or an enterprise entrusted by a corporation, etc., but it may be an enterprise that includes various organizations and institutions. In addition, the operator may also be an enterprise that operates a public or private medical insurance system, or a medical profession that provides medical services.

(10) The support services in the above-described exemplary embodiments aim to support the prevention, diagnosis, and therapy of high blood pressure, but can also be applied to other diseases.

The support services can be applied to customize messages in services that support the improvement of lifestyle related to, for example, nicotine addiction, insomnia disorder, depression, diabetes, alcoholism, and obesity.

By the way, in the case of nicotine addicted users, for example, carbon monoxide (CO) in exhaled breath, nicotine concentration in saliva, date and time of smoking, the number of cigarettes smoked or the number of times of smoking, user cognitive information (way of thinking, values), exercise (the number of steps, length of exercise time, travel distance), other diseases (diabetes), medications, blood pressure, heart rate, pulse, current physical condition (headache, irritation, feeling good, nausea, etc.), and all or part of the answers for assumed causes are stored as the user data. These information items are examples of information related to smoking.

In addition, in the case of insomnia users, all or part of, for example, bedtime, wake time, sleep hours, sleep efficiency, the number and content of meals, exercise records, and medication records is stored as the user data. The bedtime, wake time, sleep hours, and sleep efficiency are examples of information related to sleep.

In addition, in the case of depressed users, all or part of, for example, mood records, action records, medications records, sleep records, and appetite records is stored as the user data.

In addition, in the case of users with diabetes, all or part of, for example, the number and content of meals, exercise records, medication records, weight, blood pressure value, and blood sugar level is stored as the user data. The blood sugar level is an example of information about the blood sugar level.

In addition, in the case of alcoholic users, all or part of, for example, medications records, presence or absence of drinking, and content of drinking is stored as the user data.

In addition, in the case of users with obesity, all or part of, for example, a target weight, therapy start date, the number of days of therapy, optimum intake calorie, physical condition history (mood), weight history, calorie intake history, calorie intake date and time, medications history (dose, medications date and time), therapy history (therapy, details of advice, presence or absence of self-care by the user), medical care history, blood data (aspartate aminotransferase (AST), alanine aminotransferase (ALT)), and CT values is stored as the user data.

### Conclusion

The disclosure examples described in the above-described exemplary embodiments are shown below.

(((1)))

An information processing system including at least one processor, wherein the at least one processor presents at least one high-priority action category based on information about lifestyle of a target user, and presents the target user with at least one action to be practiced by the target user in relation to the presented action category.

According to the information processing system, it is possible to support improvement in effectiveness of lifestyle improvement actions.

(((2)))

The information processing system described in (((1))), wherein, when the at least one action is plural actions, the processor presents the plural actions in a selectable manner.

According to the information processing system, it is possible to increase the likelihood of actions that improve lifestyle to be practiced.

(((3)))

The information processing system described in (((1))) or (((2))), wherein the processor presents a content encouraging practice of an action selected by the target user.

According to the information processing system, it is possible to increase the likelihood of actions that improve lifestyle to be practiced.

(((4)))

The information processing system described in any one of (((1))) to (((3))), wherein the processor presents the target user with an analysis result of the information about lifestyle to the target user.

According to the information processing system, it is possible to increase the likelihood of actions that improve lifestyle to be practiced.

(((5)))

The information processing system described in (((4))), wherein, based on the analysis result and actual performance data of a user other than the target user, the processor presents the at least one high-priority action category.

According to the information processing system, it is possible to present action categories that have high effects on lifestyle improvement.

(((6)))

The information processing system described in any one of (((1))) to (((3))), wherein the processor updates the at least one action to be practiced by the target user in a cycle shorter than a cycle of updating the action category.

According to the information processing system, it is possible to increase the likelihood of actions that improve lifestyle to be practiced.

(((7)))

An information processing method including: presenting at least one high-priority action category based on information about lifestyle of a target user; and presenting the target user with at least one action to be practiced by the target user in relation to the presented action category.

According to the information processing method, it is possible to support improvement in effectiveness of lifestyle improvement actions.

Note that, in this information processing method, it is possible to combine technologies corresponding to (((2))) to (((6))).

(((8)))

A non-transitory computer readable medium storing a program that causes a computer to execute an information processing method including: presenting at least one high-priority action category based on information about lifestyle of a target user; and presenting the target user with at least one action to be practiced by the target user in relation to the presented action category.

According to the program, it is possible to support improvement in effectiveness of lifestyle improvement actions.

Note that, in this program, it is possible to combine technologies corresponding to (((2))) to (((6))).

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The exemplary embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing system comprising:
at least one processor, wherein
the at least one processor presents at least one high-priority action category based on information about lifestyle of a target user, and presents the target user with at least one action to be practiced by the target user in relation to the presented action category.

2. The information processing system according to claim 1, wherein, when the at least one action is a plurality of actions, the processor presents the plurality of actions in a selectable manner.

3. The information processing system according to claim 2, wherein the processor presents a content encouraging practice of an action selected by the target user.

4. The information processing system according to claim 1, wherein the processor presents the target user with an analysis result of the information about lifestyle to the target user.

5. The information processing system according to claim 4, wherein, based on the analysis result and actual performance data of a user other than the target user, the processor presents the at least one high-priority action category.

6. The information processing system according to claim 1, wherein the processor updates the at least one action to be practiced by the target user in a cycle shorter than a cycle of updating the action category.

7. An information processing method comprising:
presenting at least one high-priority action category based on information about lifestyle of a target user; and
presenting the target user with at least one action to be practiced by the target user in relation to the presented action category.

8. A non-transitory computer readable medium storing a program that causes a computer to execute an information processing method comprising:
presenting at least one high-priority action category based on information about lifestyle of a target user; and
presenting the target user with at least one action to be practiced by the target user in relation to the presented action category.
